(19) United States
(12) Patent Application Publication
(10) Pub. No.: US 2026/0347801 A1
(43) Pub. Date: Nov. 5, 2026

(54) GLYCOSYLTRANSFERASE FOR CATALYZING PRODUCTION OF REBAUDIOSIDE M

(71) Applicant: HONGZHOU LEVGINTHAL BIOTECHNOLOGY CO., LTD., Hangzhou (CN)

(72) Inventors: Wenhan Zhang, Hangzhou (CN); Weikun Wu, Hangzhou (CN); Dechuan Liu, Hangzhou (CN); Haobo Wang, Hangzhou (CN); Rui Ma, Hangzhou (CN); Jiawei Guan, Hangzhou (CN)

(73) Assignee: HONGZHOU LEVGINTHAL BIOTECHNOLOGY CO., LTD., Hangzhou (CN)

(21) Appl. No.: 19/682,259

(22) Filed: May 19, 2026

Related U.S. Application Data
(63) Continuation of application No. PCT/CN2025/134034, filed on Nov. 11, 2025.

(30) Foreign Application Priority Data
Apr. 23, 2025 (CN) 202510518426.4

(51) Int. Cl. C12N 9/10; C12N 15/70; C12P 19/18; C12P 19/56

(52) U.S. Cl. CPC C12N 9/1051; C12N 15/70; C12P 19/18; C12P 19/56; C12Y 204/01

(57) ABSTRACT

The present disclosure provides a glycosyltransferase for catalyzing production of Rebaudioside M (Reb M) and a recombinant gene encoding the glycosyltransferase. Compared with a wild-type glycosyltransferase, glycosyltransferase variants of the present disclosure exhibit significantly improved thermal stability and enzyme activity. At a high temperature of 70° C., the wild-type glycosyltransferase is completely inactivated, whereas the glycosyltransferase variants of the present disclosure retain a specific activity of not less than 50 U/mg. In addition, at 70° C., the solubility of the reaction substrate Rebaudioside D (Reb D) is increased, and the thermal motion of enzyme molecules and substrate molecules is intensified, thereby increasing collision frequency and collision energy between enzyme molecules and the substrate molecules and further accelerating the catalytic reaction. Accordingly, the glycosyltransferase variants of the present disclosure are suitable for use as biocatalysts for large-scale production of the sweetener Rebaudioside M (Reb M) having an improved taste profile.

The present disclosure claims priority to Chinese patent application No. 202510518426.4, filed on Apr. 23, 2025 with the China National Intellectual Property Administration and entitled “Glycosyltransferase for Catalyzing Production of Rebaudioside M and Recombinant Gene Thereof,” the entire disclosure of which is hereby incorporated by reference in its entirety.

### SEQUENCE LISTING

This application contains a sequence listing in computer readable form (File name: 19234030001.xml; date of creation: May 14, 2026; File size: 16,384 bytes) which is incorporated herein by reference in its entirety and forms part of the disclosure.

### TECHNICAL FIELD

The present disclosure relates to the field of bioengineering, and more particularly to a glycosyltransferase for catalyzing production of Rebaudioside M (Reb M) and a recombinant gene thereof.

### BACKGROUND

Steviol glycosides, also referred to as stevia glycosides, have high sweetness (300 to 450 times that of sucrose) and a low caloric value (about 1/300 that of sucrose). As a natural substitute for sucrose, they are known as the “third sugar source” worldwide. Steviol glycosides are extracted from Stevia rebaudiana, and in the steviol glycosides extracted from Stevia rebaudiana by manufacturers, stevioside (STV) and Rebaudioside A (Reb A) account for the major proportion. However, STV and Reb A have a pronounced bitterness and licorice-like aftertaste, which greatly limits the use of steviol glycosides as sweeteners in foods.

Studies have shown that Rebaudioside M (Reb M) retains the advantages of steviol glycosides in terms of high sweetness and low caloric value while exhibiting almost no bitterness or licorice-like aftertaste, and thus is a superior sugar substitute. However, there are significant bottlenecks in large-scale production of Reb M. The content of Reb M in Stevia rebaudiana is less than 1%, and production of Reb M by plant extraction is costly. In addition, glycosyltransferases currently used to catalyze conversion of Rebaudioside D (Reb D) to Reb M exhibit relatively low catalytic specific activity, resulting in low production efficiency of Reb M. As a result, the cost of producing Reb M by microbial fermentation or enzymatic catalysis remains relatively high. Therefore, how to improve the specific activity of glycosyltransferases is an urgent technical problem to be solved.

### SUMMARY

A glycosyltransferase can transfer a glucose moiety onto Rebaudioside D (Reb D) to generate Rebaudioside M (Reb M). However, the substrate Rebaudioside D (Reb D) has poor solubility at ambient temperature. For example, at room temperature (25° C.), its solubility is only about 300 ppm (i.e., 0.03 wt %), resulting in very low reaction efficiency. In contrast, increasing the temperature (to 70° C. and above) can greatly enhance the solubility of Reb D to about 2.5%, which is favorable for carrying out the reaction and improving the yield of Reb M.

In view of the foregoing, one object of the present disclosure is to provide a glycosyltransferase variant having an optimum reaction temperature of about 70° C. and a specific activity of greater than 40 U/mg.

In order to achieve the foregoing object, the present disclosure provides the following technical solution. A thermostable glycosyltransferase is provided, whose wild-type sequence is shown in SEQ ID NO: 1. The wild-type glycosyltransferase sequence is derived from protein sequence library entry GI:1777435608 and is a glycosyltransferase originating from Stevia rebaudiana. Its amino acid sequence is as follows:

(SEQ ID NO: 1)
MMENKTETTVRRRRRIILFPVPFQGHINPILQLANVLYSKGFSITIFHTN
FNKPKTSNYPHFTFRFILDNDPQDERISNLPTHGPLAGMRIPIINEHGAD
ELRRELELLMLASEEDEEVSCLITDALWYFAQSVADSLNLRRLVLMTSSL
FNFHAHVSLPQFDELGYLDPDDKTRLEEQASGFPMLKVKDIKSAYSNWQI
LKEILGKMIKQTKASSGVIWNSFKELEESELETVIREIPAPSFLIPLPKH
LTASSSSLLDHDRTVFQWLDQQPPSSVLYVSFGSTSEVDEKDFLEIARGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRRVMVDEEGEYIRQNARVLKQKADVSLMKGGSSYESLE
SLVSYISSLTS.

By using the Lésign platform, sequence design was performed on the wild-type glycosyltransferase to obtain enzyme variants that were computationally predicted to be optimal. Approximately 30 predicted variants were individually expressed and evaluated for specific activity. Some variants exhibited performance at elevated temperatures comparable to that of the wild-type enzyme, some performed worse than the wild-type, some were slightly better, and some were significantly better. Ultimately, eight variants with high specific activity at elevated temperatures were obtained.

A glycosyltransferase for catalyzing production of Rebaudioside M (Reb M), wherein the glycosyltransferase is derived from the wild-type glycosyltransferase of SEQ ID NO: 1 and comprises the following set of mutations: K55N+N70T+S181P+S275R+K441E+T460K+S193R+M146R+E3V, which variant is referred to as Variant 1, and its amino acid sequence is as follows:

(SEQ ID NO: 2)
MMVNKTETTVRRRRRIILFPVPFQGHINPILQLANVLYSKGFSITIFHTN
FNKPNTSNYPHFTFRFILDTDPQDERISNLPTHGPLAGMRIPIINEHGAD
ELRRELELLMLASEEDEEVSCLITDALWYFAQSVADSLNLRRLVLRTSSL
FNFHAHVSLPQFDELGYLDPDDKTRLEEQAPGFPMLKVKDIKRAYSNWQI
LKEILGKMIKQTKASSGVIWNSFKELEESELETVIREIPAPSFLIPLPKH
LTASSSSLLDHDRTVFQWLDQQPPRSVLYVSFGSTSEVDEKDFLEIARGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRRVMVDEEGEYIRQNARVLKQKADVSLMEGGSSYESLE
SLVSYISSLKS.

A second object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), wherein the glycosyltransferase is derived from the wild-type glycosyltransferase of SEQ ID NO: 1 and comprises the following set of mutations: K55N+N70T+S181P+S275R+K441E+T460K+M146R+D327E+K224E, which variant is referred to as Variant 2, and its amino acid sequence is as follows:

(SEQ ID NO: 3)
MMENKTETTVRRRRRIILFPVPFQGHINPILQLANVLYSKGFSITIFHTN
FNKPNTSNYPHFTFRFILDTDPQDERISNLPTHGPLAGMRIPIINEHGAD
ELRRELELLMLASEEDEEVSCLITDALWYFAQSVADSLNLRRLVLRTSSL
FNFHAHVSLPQFDELGYLDPDDKTRLEEQAPGFPMLKVKDIKSAYSNWQI
LKEILGKMIKQTKASSGVIWNSFEELEESELETVIREIPAPSFLIPLPKH
LTASSSSLLDHDRTVFQWLDQQPPRSVLYVSFGSTSEVDEKDFLEIARGL
VDSKQSFLWVVRPGFVKGSTWVEPLPEGFLGERGRIVKWVPQQEVLAHGA
IGAFWTHSGWNSTLESVCEGVPMIFSDFGLDQPLNARYMSDVLKVGVYLE
NGWERGEIANAIRRVMVDEEGEYIRQNARVLKQKADVSLMEGGSSYESLE
SLVSYISSLKS.

A third object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), the glycosyltransferase being derived from the wild-type glycosyltransferase of SEQ ID NO: 1 and comprising the following set of mutations: K55N+N70T+S181P+S275R+K441E+T460K+K224E+I94F+R414H+A112S, which variant is referred to as Variant 3, and its amino acid sequence is as follows:

(SEQ ID NO: 4)
MMENKTETTVRRRRRIILFPVPFQGHINPILQLANVLYSKGFSITIFHTN
FNKPNTSNYPHFTFRFILDTDPQDERISNLPTHGPLAGMRIPIFNEHGAD
ELRRELELLMLSSEEDEEVSCLITDALWYFAQSVADSLNLRRLVLMTSSL
FNFHAHVSLPQFDELGYLDPDDKTRLEEQAPGFPMLKVKDIKSAYSNWQI
LKEILGKMIKQTKASSGVIWNSFEELEESELETVIREIPAPSFLIPLPKH
LTASSSSLLDHDRTVFQWLDQQPPRSVLYVSFGSTSEVDEKDFLEIARGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRHVMVDEEGEYIRQNARVLKQKADVSLMEGGSSYESLE
SLVSYISSLKS.

A fourth object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), the glycosyltransferase being derived from the wild-type glycosyltransferase of SEQ ID NO: 1 and comprising the following set of mutations: K55N+N70T+S181P+S275R+K441E+T460K+194F+R90L+S242I+H156Y+A180V+R175H, which variant is referred to as Variant 4, and its amino acid sequence is as follows:

(SEQ ID NO: 5)
MMENKTETTVRRRRRIILFPVPFQGHINPILQLANVLYSKGFSITIFHTN
FNKPNTSNYPHFTFRFILDTDPQDERISNLPTHGPLAGMLIPIFNEHGAD
ELRRELELLMLASEEDEEVSCLITDALWYFAQSVADSLNLRRLVLMTSSL
FNFHAYVSLPQFDELGYLDPDDKTHLEEQVPGFPMLKVKDIKSAYSNWQI
LKEILGKMIKQTKASSGVIWNSFKELEESELETVIREIPAPIFLIPLPKH
LTASSSSLLDHDRTVFQWLDQQPPRSVLYVSFGSTSEVDEKDFLEIARGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRRVMVDEEGEYIRQNARVLKQKADVSLMEGGSSYESLE
SLVSYISSLKS.

A fifth object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), the glycosyltransferase being derived from the wild-type glycosyltransferase of SEQ ID NO: 1 and comprising the following set of mutations: K55N+N70T+S181P+S275R+K441E+T460K+E3V+R414H+K224E+A112S+R90L+M146R+R29 8Q+A180V, which variant is referred to as Variant 5, and its amino acid sequence is as follows:

(SEQ ID NO: 6)
MMVNKTETTVRRRRRIILFPVPFQGHINPILQLANVLYSKGFSITIFHTN
FNKPNTSNYPHFTFRFILDTDPQDERISNLPTHGPLAGMLIPIINEHGAD
ELRRELELLMLSSEEDEEVSCLITDALWYFAQSVADSLNLRRLVLRTSSL
FNFHAHVSLPQFDELGYLDPDDKTRLEEQVPGFPMLKVKDIKSAYSNWQI
LKEILGKMIKQTKASSGVIWNSFEELEESELETVIREIPAPSFLIPLPKH
LTASSSSLLDHDRTVFQWLDQQPPRSVLYVSFGSTSEVDEKDFLEIAQGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRHVMVDEEGEYIRQNARVLKQKADVSLMEGGSSYESLE
SLVSYISSLKS.

A sixth object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), the glycosyltransferase being derived from the wild-type glycosyltransferase of SEQ ID NO: 1 and comprising the following set of mutations: K55N+N70T+S181P+S275R+K441E+T460K+S193R+D100A+R298Q+K224E+R141P+R414H, which variant is referred to as Variant 6, and its amino acid sequence is as follows:

(SEQ ID NO: 7)
MMENKTETTVRRRRRIILFPVPFQGHINPILQLANVLYSKGFSITIFHTN
FNKPNTSNYPHFTFRFILDTDPQDERISNLPTHGPLAGMRIPIINEHGAA
ELRRELELLMLASEEDEEVSCLITDALWYFAQSVADSLNLPRLVLMTSSL
FNFHAHVSLPQFDELGYLDPDDKTRLEEQAPGFPMLKVKDIKRAYSNWQI
LKEILGKMIKQTKASSGVIWNSFEELEESELETVIREIPAPSFLIPLPKH
LTASSSSLLDHDRTVFQWLDQQPPRSVLYVSFGSTSEVDEKDFLEIAQGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRHVMVDEEGEYIRQNARVLKQKADVSLMEGGSSYESLE
SLVSYISSLKS.

A seventh object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), the glycosyltransferase being derived from the wild-type glycosyltransferase of SEQ ID NO: 1 and comprising the following set of mutations: K55N+N70T+S181P+S275R+K441E+T460K+D100A+R414H+K224E+R405P+T264S+194F+R9 0L, which variant is referred to as Variant 7, and its amino acid sequence is as follows:

(SEQ ID NO: 8)
MMENKTETTVRRRRRIILFPVPFQGHINPILQLANVLYSKGFSITIFHTN
FNKPNTSNYPHFTFRFILDTDPQDERISNLPTHGPLAGMLIPIFNEHGAA
ELRRELELLMLASEEDEEVSCLITDALWYFAQSVADSLNLRRLVLMTSSL
FNFHAHVSLPQFDELGYLDPDDKTRLEEQAPGFPMLKVKDIKSAYSNWQI
LKEILGKMIKQTKASSGVIWNSFEELEESELETVIREIPAPSFLIPLPKH
LTASSSSLLDHDRSVFQWLDQQPPRSVLYVSFGSTSEVDEKDFLEIARGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWEPGEIANAIRHVMVDEEGEYIRQNARVLKQKADVSLMEGGSSYESLE
SLVSYISSLKS.

An eighth object of the present disclosure is to provide another glycosyltransferase variant having an optimum reaction temperature of about 70° C. and a specific activity of greater than 40 U/mg.

In order to achieve the foregoing object, the present disclosure provides the following technical solution. A glycosyltransferase for catalyzing production of Rebaudioside M (Reb M), the glycosyltransferase being derived from the wild-type glycosyltransferase of SEQ ID NO: 1 and comprising the following set of mutations: K55N+N70T+S181P+S275R+K441E+T460K+R414H+R90L+A112S+N50K+S193R+E3V+H156 Y+T264S+R405P+D327E, which variant is referred to as Variant 8, and its amino acid sequence is as follows:

(SEQ ID NO: 9)
MMVNKTETTVRRRRRIILFPVPFQGHINPILQLANVLYSKGFSITIFHTK
FNKPNTSNYPHFTFRFILDTDPQDERISNLPTHGPLAGMLIPIINEHGAD
ELRRELELLMLSSEEDEEVSCLITDALWYFAQSVADSLNLRRLVLMTSSL
FNFHAYVSLPQFDELGYLDPDDKTRLEEQAPGFPMLKVKDIKRAYSNWQI
LKEILGKMIKQTKASSGVIWNSFKELEESELETVIREIPAPSFLIPLPKH
LTASSSSLLDHDRSVFQWLDQQPPRSVLYVSFGSTSEVDEKDFLEIARGL
VDSKQSFLWVVRPGFVKGSTWVEPLPEGFLGERGRIVKWVPQQEVLAHGA
IGAFWTHSGWNSTLESVCEGVPMIFSDFGLDQPLNARYMSDVLKVGVYLE
NGWEPGEIANAIRHVMVDEEGEYIRQNARVLKQKADVSLMEGGSSYESLE
SLVSYISSLKS.

The inventors analyzed the mutation sites in the above eight variants. The results are shown in Table 1.

TABLE 1
Mutation sites of the eight variants

| Mutation Site | Wild-type | Variant | | | | | | | |
|---|---|---|---|---|---|---|---|---|---|
| (Position) | amino acid | 1 | 2 | 3 | 4 | 5 | 6 | 7 | 8 |
| 55 | K | N | N | N | N | N | N | N | N |
| 70 | N | T | T | T | T | T | T | T | T |
| 181 | S | P | P | P | P | P | P | P | P |
| [symbol] | S | R | R | R | R | R | R | R | R |
| 441 | K | E | E | E | E | E | E | E | E |
| 460 | T | K | K | K | K | K | K | K | K |
| 224 | K | | E | E | | E | E | E | |
| 414 | R | | | H | | H | H | H | H |
| [symbol] | R | | | | L | L | | L | L |
| 3 | E | V | | | | V | | | V |
| 94 | I | | | F | F | | | F | |
| 112 | A | | | S | | S | | | S |
| [symbol] | M | R | R | | | R | | | |
| 193 | S | R | | | | | R | | R |
| 100 | D | | | | | | A | A | |
| 156 | N | | | | Y | | | | Y |
| 180 | A | | | | V | V | | | |
| 264 | T | | | | | | | S | S |
| 298 | R | | | | | [symbol] | [symbol] | | |
| 327 | O | | E | | | | | | E |
| 405 | R | | | | | | | P | P |
| 50 | N | | | | | | | | K |
| 141 | R | | | | | | P | | |
| 175 | R | | | | H | | | | |
| 242 | S | | | | I | | | | |

[symbol]

indicates data missing or illegible when filed

The inventors unexpectedly found that these variants share a common technical feature, namely that each of the variants comprises the mutation combination K55N, N70T, S181P, S275R, K441E, and T460K (K55N+N70T+S181P+S275R+K441E+T460K). It was verified that a variant containing only this set of six mutation sites exhibits an activity in the temperature range of 50° C. to 75° C. that is significantly higher than that of the wild-type glycosyltransferase. On the basis of this combination of six mutation sites, introduction of additional mutation sites can still maintain good specific activity at elevated temperatures, as in Variants 1-8.

The present disclosure provides a glycosyltransferase variant for catalyzing production of Rebaudioside M (Reb M), wherein, compared with the wild-type glycosyltransferase shown in SEQ ID NO: 1, the glycosyltransferase variant comprises a combination of mutation sites K55N+N70T+S181P+S275R+K441E+T460K. In some embodiments, the glycosyltransferase variant has a mutation combination K55N+N70T+S181P+S275R+K441E+T460K+S193R+M146R+E3V, and has an amino acid sequence as set forth in SEQ ID NO: 2. In other embodiments, the glycosyltransferase variant has a mutation combination K55N+N70T+S181P+S275R+K441E+T460K+M146R+D327E+K224E, and has an amino acid sequence as set forth in SEQ ID NO: 3. In yet other embodiments, the glycosyltransferase variant has a mutation combination K55N+N70T+S181P+S275R+K441E+T460K+K224E+194F+R414H+A112S, and has an amino acid sequence as set forth in SEQ ID NO: 4. In yet other embodiments, the glycosyltransferase variant has a mutation combination K55N+N70T+S181P+S275R+K441E+T460K+194F+R90L+S242I+H156Y+A180V+R175H, and has an amino acid sequence as set forth in SEQ ID NO: 5. In yet other embodiments, the glycosyltransferase variant has a mutation combination K55N+N70T+S181P+S275R+K441E+T460K+E3V+R414H+K224E+A112S+R90L+M146R+R29 8Q+A180V, and has an amino acid sequence as set forth in SEQ ID NO: 6. In yet other embodiments, the glycosyltransferase variant has a mutation combination K55N+N70T+S181P+S275R+K441E+T460K+S193R+D100A+R298Q+K224E+R141P+R414H, and has an amino acid sequence as set forth in SEQ ID NO: 7. In yet other embodiments, the glycosyltransferase variant has a mutation combination K55N+N70T+S181P+S275R+K441E+T460K+D100A+R414H+K224E+R405P+T264S+194F+R9 0L, and has an amino acid sequence as set forth in SEQ ID NO: 8. In yet other embodiments, the glycosyltransferase variant has a mutation combination K55N+N70T+S181P+S275R+K441E+T460K+R414H+R90L+A112S+N50K+S193R+E3V+H156 Y+T264S+R405P+D327E, and has an amino acid sequence as set forth in SEQ ID NO: 9. In yet other embodiments, the glycosyltransferase variant has a mutation combination K55N+N70T+S181P+S275R+K441E+T460K, and has an amino acid sequence as set forth in SEQ ID NO: 10.

The present disclosure further provides a method for preparing Rebaudioside M (Reb M). The method comprises mixing a substrate Rebaudioside D (Reb D) with the glycosyltransferase variant of the present disclosure and allowing the mixture to react. The reaction is carried out at a temperature from 50° C. to 75° C., for example from 55° C. to 75° C., from 60° C. to 75° C., from 65° C. to 75° C., from 60° C. to 70° C., or from 70° C. to 75° C..

A ninth object of the present disclosure is to provide genetic material capable of expressing the above glycosyltransferase variants. To achieve this object, the present disclosure provides recombinant genetic material of a glycosyltransferase, the recombinant genetic material being DNA or RNA capable of expressing any one of the glycosyltransferase variants described above.

A tenth object of the present disclosure is to provide a recombinant strain capable of expressing the above glycosyltransferase variants. To achieve this object, the present disclosure provides a recombinant strain of a glycosyltransferase, the recombinant strain comprising recombinant genetic material capable of expressing any one of the glycosyltransferase variants described above.

Compared with the prior art, the present disclosure has the following advantages. The substrate Rebaudioside D (Reb D) and the product Rebaudioside M (Reb M) catalyzed by the enzyme have poor solubility at low temperatures, whereas increasing the temperature can greatly enhance their solubility and is favorable for the reaction to proceed.

Compared with the wild-type glycosyltransferase, the glycosyltransferase variants of the present disclosure exhibit significantly improved thermal stability and specific activity. At a high temperature of 70° C., the wild-type glycosyltransferase is completely inactivated, whereas the glycosyltransferase variants of the present disclosure retain a specific activity of at least 50 U/mg. At the same time, at 70° C., the solubility of the reaction substrate Rebaudioside D (Reb D) is increased and the thermal motion of enzyme molecules and substrate molecules is intensified, thereby increasing the collision frequency and collision energy between enzyme molecules and substrate molecules and further accelerating the catalytic reaction. Accordingly, the glycosyltransferase variants of the present disclosure are suitable for use as biocatalysts for large-scale production of the sweetener Rebaudioside M (Reb M) having an improved taste profile.

### DETAILED DESCRIPTION

The present disclosure provides a glycosyltransferase for catalyzing production of Rebaudioside M (Reb M) and a recombinant gene thereof. Those skilled in the art can refer to the disclosure herein and implement the present disclosure by appropriately adjusting process parameters. It is to be particularly noted that all similar substitutions and modifications are apparent to those skilled in the art and are deemed to be included within the scope of the present disclosure. The methods and uses of the present disclosure have been described by way of preferred embodiments, and it is apparent to those skilled in the art that various modifications, appropriate changes, and combinations of the methods and uses described herein may be made without departing from the substance, spirit, and scope of the present disclosure.

Unless otherwise indicated, all technical and scientific terms used herein have the same meaning as commonly understood by the person skilled in the art to which the present invention belongs.

As used herein, the following terms have the meanings ascribed to them below, unless the context clearly indicates otherwise.

### DEFINITIONS

The term “recombinant gene” refers to DNA or RNA that is capable of expressing the glycosyltransferase of the present disclosure. Typically, a recombinant gene is initially synthesized in vitro by a solid-phase phosphoramidite triester method, a TdT-based biosynthetic method, or other suitable techniques known in the art. Once a template sequence is available, the recombinant gene may be amplified by PCR or other suitable amplification techniques known in the art. After a recombinant strain has been obtained, further large-scale amplification may be achieved by culturing the strain. In some embodiments, the recombinant gene may further include restriction site scar sequences and other ancillary elements, such as control elements (e.g., promoters), markers (e.g., fluorescent labels), and other sequences that do not adversely affect expression of the gene of interest.

The term “cloning scar” refers to a situation in which protein expression depends on a promoter sequence for initiating transcription of an initial messenger ribonucleic acid (mRNA), followed by a ribosome binding site (RBS) that recruits the translation machinery, and then a signal peptide sequence that facilitates transport of the protein to the periplasm. A mature protein is typically cloned downstream of the signal peptide, and as the mature protein passes through the membrane, the signal peptide is cleaved from the mature protein by a signal peptidase. However, when a construct is cloned downstream of the signal peptide, restriction endonucleases usually require specific sequences in order to cut the DNA, which leaves a cloning scar immediately after the signal peptide sequence.

The term “signal peptide” refers to a short peptide (typically 16 to 30 amino acids in length) located at the N-terminus of most newly synthesized proteins that are destined to enter the secretory pathway. It may also be referred to as a signal sequence, targeting signal, localization signal, localization sequence, transit peptide, leader sequence, or leader peptide. A signal peptide is typically cleaved from the protein by a signal peptidase.

Regardless of whether a cloning scar, a signal peptide, or other elements in the recombinant gene are present, such elements do not affect realization of the function of the glycosyltransferase. Accordingly, if the amino acid sequence of a final protein differs from an amino acid sequence disclosed in the present disclosure only in amino acid residues corresponding to the DNA sequences of the foregoing elements, the protein still falls within the scope of protection of the present disclosure.

The term “signal peptide cleavage site” refers to a dipeptide at which a signal peptidase cleaves the signal peptide from the mature protein, namely between the two amino-acid residues of the dipeptide. In most, but not all, cases the dipeptide is Ala-Ala. The signal peptide cleavage site can be predicted using algorithms such as SignalP 4.1, which is available online at http://www.cbs.dtu.dk/services/SignalP/ (Center for Biological Sequence Analysis, Technical University of Denmark).

The term “promoter” refers to a DNA region that initiates transcription of a particular gene into mRNA. A promoter is typically located near the transcription start site of the gene, on the same strand and upstream on the DNA (toward the 5′ region of the sense strand). A promoter may be inducible, meaning that expression of a gene operably linked to the promoter can be turned on in the presence of an inducer substance. Alternatively, a promoter may be constitutive, meaning that it is not regulated by any inducer substance.

The abbreviation “RBS” refers to a ribosome binding site, i.e., a binding site for the ribosome. The ribosome binding site is a nucleotide sequence located upstream of the start codon in an mRNA transcript and is responsible for recruiting the ribosome during initiation of protein translation.

The term “expression” refers to the process in which DNA is transcribed into messenger RNA (mRNA) and subsequently translated into a protein. To achieve efficient expression and screening of the glycosyltransferase, a signal peptide, a promoter, an RBS, or other such elements may be introduced into the recombinant gene, and as a result, portions of the corresponding peptide sequences may remain on the expressed glycosyltransferase protein. These peptide sequences do not affect the function of the glycosyltransferase; therefore, even if such peptide sequences are present in the product, the product is still considered falls within the scope of the present disclosure so long as the main portion of the amino acid sequence is identical to a sequence of the present disclosure.

The term “expression vector” refers to a vector capable of incorporating and expressing a heterologous polynucleotide sequence in a host cell. Many prokaryotic and eukaryotic expression vectors are commercially available. Selection of an appropriate expression vector is within the knowledge of those skilled in the art.

The term “chassis cell” refers to a suitable host for expressing a vector containing the DNA of the present disclosure. The host may be any organism capable of containing and expressing the nucleic acid or gene disclosed herein, without limitation. The chassis cell may be prokaryotic or eukaryotic, unicellular or multicellular, including, for example, mammalian cells, plant cells, fungi, and the like. In accordance with the prior art, those skilled in the art can achieve heterologous expression of the recombinant DNA of the present disclosure in different known chassis cells by adjusting parameters through a limited number of experiments.

The chassis cell may be selected from at least one of Escherichia coli, Pichia pastoris, Saccharomyces cerevisiae, Hansenula polymorpha, Candida spp., Rhodotorula spp., Bacillus spp., Escherichia spp., Salmonella spp., Clostridium spp., Streptomyces spp., Staphylococcus spp., Neisseria spp., and Shigella spp. The foregoing enumeration of chassis cell types is merely illustrative and is not intended to limit the types of chassis cells.

The chassis cell is preferably Escherichia coli. Suitable E. coli strains (including many other strains) include BL21 (DE3), C600, DH5αF′, 1113101, JM83, JM101, JM103, JM105, JM107, JM109, JM110, MC1061, MC4100, MM294, NM522, NM554, TGI, χ1776, XL1-Blue, and Y1089⁺, among others. The above E. coli strains are all commercially available.

The term “identity” means that residues in two sequences are the same at corresponding positions when the sequences are aligned for maximal correspondence, as measured using sequence comparison or analysis algorithms such as those described herein. For example, if upon proper alignment, corresponding segments of two sequences have the same residue at 5 out of 10 positions, the two sequences are said to have 50% identity. Most bioinformatics programs report the percentage identity of an aligned region of the sequences, which region is typically less than the full length of the molecules. If the alignment is sufficiently long and contains a sufficient number of identical residues, an expectation value can be calculated, indicating that the observed level of identity in the alignment is unlikely to have occurred by chance.

The raw materials and reagents used in the glycosyltransferase for catalyzing production of Rebaudioside M (Reb M) and the recombinant gene thereof provided by the present disclosure are all commercially available. The present disclosure will be further illustrated below with reference to the following examples.

### Example 1

Proteins are the material basis of life and are important components of human cells and tissues. All important constituents of the human body require the participation of proteins, which play a crucial role in the life activities of cells and organisms. It can be said that without proteins, there is no life. There are numerous types of proteins in the human body, and their functions vary. Some proteins constitute body tissues, some provide energy, some participate in metabolism and substance transport, and some promote growth and development or regulate immune functions. Different proteins undertake different roles and functions, and their functions are determined by their structures. The three-dimensional structure of a protein is in turn determined by its amino acid sequence. Therefore, protein design relies on the correspondence between structure and sequence. To design a protein with a specific function, it is necessary to design a sequence that gives rise to a structure consistent with that function. Understanding and designing proteins is of great significance for promoting innovation and advances in biology and medicine.

Designing a protein sequence for a particular function is a highly challenging task, and the final structure and function exhibited by a designed sequence are difficult to predict. In addition, the sample space of protein sequences of a fixed length is extremely large. To accomplish the foregoing work, Liwen Institute has developed a deep-learning-based protein design platform, Lésign. This platform implements functions such as protein structure prediction, sequence design, and result evaluation. The individual functional modules cooperate with one another through interfaces to form an integrated computational pipeline that combines prediction, design, and evaluation.

Using, as an example, a glycosyltransferase derived from the plant Stevia rebaudiana, sequence design was carried out on the wild-type glycosyltransferase (having an amino acid sequence as shown in SEQ ID NO: 1) by means of the Lésign platform to obtain enzyme variants that are computationally predicted to be optimal. Approximately 30 predicted variants were individually expressed and evaluated for specific activity, and ultimately eight variants were obtained that still exhibit high specific activity at elevated temperatures.

### 1. Preparation of Glycosyltransferase

1.1 Construction of recombinant cells

The nucleotide sequences of the genes of interest were synthesized by Beijing Qingke Biotechnology Co., Ltd., and the nucleotide sequence encoding the glycosyltransferase was inserted into an expression vector. Specifically, the nucleotide sequence was inserted into the plasmid pET-28a(+), thereby obtaining the corresponding plasmids. The synthesized plasmids were then introduced into chassis cells (E. coli BL21 (DE3)) to construct Escherichia coli strains containing the plasmids. Many other plasmids and chassis cells known in the art may also be used; the present embodiment merely provides one specific scheme.
1.2 Expression and purification of glycosyltransferase

The recombinant strain was inoculated at 1% (v/v) into a shake flask and cultured overnight. The overnight culture was then inoculated at 1% (v/v) into a fermenter containing TB liquid medium. When the culture reached an OD₆₀₀ of about 6.0, IPTG was added to a final concentration of 100 μM, and the temperature was lowered to 16° C., followed by overnight culture. After the overnight culture, the cells were collected by centrifugation at 4000 g for 10 min and washed with 0.1 M phosphate buffer (PB, pH 7.0). The cells were then disrupted by ultrasonication, followed by centrifugation at 12000 rpm for 1 h, and the supernatant was collected as a crude enzyme solution. Numerous other methods for inducing expression of recombinant strains are known in the art; the present embodiment merely provides one specific scheme.
1.3 The crude enzyme solution was passed through a nickel affinity column for protein purification. The crude enzyme solution was eluted under different elution conditions to obtain the target protein. In accordance with the prior art, those skilled in the art can achieve purification of the glycosyltransferase by adjusting parameters through a limited number of experiments, and a detailed description is therefore omitted herein. Many other methods for purifying glycosyltransferases are also known in the art; the present embodiment merely provides one specific scheme.

The wild-type glycosyltransferase (ORM, SEQ ID NO: 1) and the glycosyltransferase variants were heterologously expressed and purified according to the above-described preparation method. The obtained glycosyltransferase and variants were verified by gel electrophoresis (SDS-PAGE) to confirm that their sizes (molecular weights) were consistent with the expected values.

### 2. Enzyme Activity Assay

2.1 Catalytic reaction system: The total volume of the enzymatic activity assay reaction system was 0.5 mL. The system contained 10 mM substrate Rebaudioside D (Reb D), 0.002 mg/mL glycosyltransferase or glycosyltransferase variant, 1 mM uridine diphosphate glucose (UDP-glucose, UDP-G) or adenosine diphosphate glucose (ADP-glucose, ADP-G) or guanosine diphosphate glucose (GDP-glucose, GDP-G) or cytidine diphosphate glucose (CDP-glucose, CDP-G), and 3 mM magnesium chloride. Glycine-sodium hydroxide buffer (0.1 M, pH 9.0) was added to bring the total volume to 0.5 mL.
2.2 Method for determining optimum reaction temperature:

The reactions were carried out according to the catalytic reaction system described in section 2.1. For the wild-type glycosyltransferase, the reaction temperatures were 25° C., 30 °C, 35° C., 40 °C, 45° C., 50 °C, 55° C., and 60° C.. For the glycosyltransferase variants, the reaction temperatures were 50° C., 55 °C, 60° C., 65 °C, 70° C., and 75° C.. In all cases, the reaction time was 1 h, and the reactions were conducted at 1000 rpm.

After completion of the reaction, the concentration of Rebaudioside M (Reb M) in the reaction mixture was determined by HPLC using an LC-2030C HT system (Shimadzu, Japan). The mobile phase consisted of acetonitrile (CH3CN, containing 0.1% formic acid (HCOOH)) and water (containing 0.1% HCOOH), with the following CH3CN gradient: 0-3 min, 25%; 3-8 min, 25-50%; 8-12 min, 50-100%; 12-15 min, 100%; and 15-20 min, 25%. The flow rate was 1 mL/min, the detection wavelength was 210 nm, and a Shim-pack GIST C18 column (250×4.6 mm, 5 μm) was used at a column temperature of 30° C.. An HPLC chromatogram was recorded, and the concentration of Reb M was calculated from the peak area.

Enzyme activity is defined as the amount of enzyme required to catalyze the formation of 1 μmol of Rebaudioside M (Reb M) per minute, and the unit is U. The specific activity of the enzyme is defined as the number of units of enzyme activity per milligram of enzyme protein, with units of U/mg (also written as μmol·min⁻¹·mg⁻¹). The specific activities of the wild-type glycosyltransferase and the glycosyltransferase variants determined by this method are shown in Tables 2-10.

TABLE 2
Specific activity of the wild-type glycosyltransferase at
different reaction temperatures

| Glycosyltransferase | Reaction | Specific activity |
|---|---|---|
| sequence | temperature | (U/mg) |
| ORM-1 (SEQ ID NO. 1) | 25 °C | 0.2369 |
| | 30 °C | 0.2400 |
| | 35 °C | 0.2381 |
| | 40 °C | 0.2222 |
| | 45 °C | 0.1877 |
| | 50 °C | 0.1421 |
| | 55 °C | 0.0523 |
| | 60 °C | 0 |

TABLE 3
Specific activity of glycosyltransferase variant
ORM-2 at different reaction temperatures

| | | Specific |
|---|---|---|
| Glycosyltransferase | Reaction | activity |
| sequence | temperature | (U/mg) |
| ORM-2 (K55N + N70T + S181P + | 50 °C | 24.81 |
| S275R + K441E + T460K + | 55 °C | 33.81 |
| S193R + M146R + E3V) | 60 °C | 41.71 |
| | 65 °C | 49.72 |
| | 70 °C | 52.67 |
| | 75 °C | 47.67 |

TABLE 4
Specific activity of glycosyltransferase variant
ORM-3 at different reaction temperatures

| [symbol] | [symbol] | [symbol] (U/mg) |
|---|---|---|
| ORM-3 | 50 °C | 20.65 |
| (K55N + N70T + S181P + S275R + | 55 °C | 24.83 |
| K441E+ T460K + M146R + | 60 °C | 32.56 |
| D327E + K224E) | 65 °C | 43.35 |
| | 70 °C | 50.23 |
| | 75 °C | 40.68 |

TABLE 5
Specific activity of glycosyltransferase variant
ORM-4 at different reaction temperatures

| Glycosyltransferase | Reaction | Specific activity |
|---|---|---|
| sequence | temperature | (U/mg) |
| ORM-4 | 50 °C | 22.23 |
| (K55N + N70T + S181P + S275R + | 55 °C | 33.56 |
| K441E + T460K + K224E + | 60 °C | 43.74 |
| 194F + R414H + A112S) | 65 °C | 51.96 |
| | 70 °C | 55.63 |
| | 75 °C | 44.42 |

TABLE 6
Specific activity of glycosyltransferase variant
ORM-5 at different reaction temperatures

| Glycosyltransferase | Reaction | Specific activity |
|---|---|---|
| sequence | temperature | (U/mg) |
| ORM-5 | 50 °C | 18.74 |
| (K55N + N70T + S181P + S275R + | 55 °C | 24.53 |
| K441E + T460K + 194F + R90L + | 60 °C | 35.86 |
| S242I + H156Y + A180V + R175H) | 65 °C | 48.51 |
| | 70 °C | 54.21 |
| | 75 °C | 42.12 |

TABLE 7
Specific activity of glycosyltransferase variant
ORM-6 at different reaction temperatures

| Glycosyltransferase | Reaction | Specific activity |
|---|---|---|
| sequence | temperature | (U/mg) |
| ORM-6 | 50 °C | 35.32 |
| (K55N + N70T + S181P + S275R + | 55 °C | 47.23 |
| K441E + T460K + E3V + R414H + | 60 °C | 51.74 |
| K224E + A112S + R90L + | 65 °C | 54.12 |
| M146R + R298Q + A180V) | 70 °C | 58.32 |
| | 75 °C | 43.21 |

TABLE 8
Specific activity of glycosyltransferase variant
ORM-7 at different reaction temperatures

| Glycosyltransferase | Reaction | Specific activity |
|---|---|---|
| sequence | temperature | (U/mg) |
| ORM-7 | 50 °C | 35.28 |
| (K55N + N70T + S181P + S275R + | 55 °C | 41.46 |
| K441E + T460K + S193R + D100A + | 60 °C | 43.24 |
| R298Q + K224E + R141P + R414H) | 65 °C | 49.13 |
| | 70 °C | 52.44 |
| | 75 °C | 47.23 |

TABLE 9
Specific activity of glycosyltransferase variant
ORM-8 at different reaction temperatures

| Glycosyltransferase | Reaction | Specific activity |
|---|---|---|
| sequence | temperature | (U/mg) |
| ORM-8 | 50 °C | 36.29 |
| (K55N + N70T + S181P + | 55 °C | 41.26 |
| S275R + K441E + T460K + | 60 °C | 50.57 |
| D100A + R414H + K224E + | 65 °C | 54.35 |
| R405P + T264S + 194F + R90L) | 70 °C | 62.79 |
| | 75 °C | 55.03 |

TABLE 10
Specific activity of glycosyltransferase variant
ORM-9 at different reaction temperatures

| | Reaction | Specific activity |
|---|---|---|
| Glycosyltransferase sequence | temperature | (U/mg) |
| ORM-9 | 50 °C | 38.43 |
| (K55N + N70T + S181P + | 55 °C | 44.14 |
| S275R + K441E + T460K + | | |
| R414H + R90L + A112S + | 60 °C | 55.83 |
| N50K + S193R + E3V + H156Y + | 65 °C | 60.98 |
| T264S + R405P + D327E) | 70 °C | 71.49 |
| | 75 °C | 58.83 |

A glycosyltransferase variant containing only the combination of mutation sites K55N+N70T+S181P+S275R+K441E+T460K was constructed using the same method, and its amino acid sequence is as follows:

(SEQ ID NO: 10)
MMENKTETTVRRRRRIILFPVPFQGHINPILQLANVLYSKGFSITIFHTN
FNKPNTSNYPHFTFRFILDTDPQDERISNLPTHGPLAGMRIPIINEHGAD
ELRRELELLMLASEEDEEVSCLITDALWYFAQSVADSLNLRRLVLMTSSL
FNFHAHVSLPQFDELGYLDPDDKTRLEEQAPGFPMLKVKDIKSAYSNWQI
LKEILGKMIKQTKASSGVIWNSFKELEESELETVIREIPAPSFLIPLPKH
LTASSSSLLDHDRTVFQWLDQQPPRSVLYVSFGSTSEVDEKDFLEIARGL
VDSKQSFLWVVRPGFVKGSTWVEPLPDGFLGERGRIVKWVPQQEVLAHGA
IGAFWTHSGWNSTLESVCEGVPMIFSDFGLDQPLNARYMSDVLKVGVYLE
NGWERGEIANAIRRVMVDEEGEYIRQNARVLKQKADVSLMEGGSSYESLE
SLVSYISSLKS.

The specific activity of the enzyme was determined in the same manner, and the results are shown in Table 11.

TABLE 11

| | Reaction | Specific activity |
|---|---|---|
| Glycosyltransferase sequence | temperature | (U/mg) |
| K55N + N70T + S181P + | 50 °C | 17.34 |
| S275R + K441E + T460K | 55 °C | 26.36 |
| | 60 °C | 37.19 |
| | 65 °C | 40.76 |
| | 70 °C | 45.45 |
| | 75 °C | 36.24 |

From Tables 2-11 it can be seen that the wild-type glycosyltransferase has an optimum reaction temperature of about 30° C. and exhibits poor thermal stability. When the reaction temperature reaches 50° C., its specific activity is only about 60% of the maximum value, and when the temperature reaches 60° C., the enzyme is completely inactivated. Even at its optimum reaction temperature, the specific activity of the wild-type glycosyltransferase is low, at only 0.24 U/mg, which is far below the level required for industrial production. In contrast, the glycosyltransferase variants provided by the present disclosure have an optimum reaction temperature of about 70° C. and exhibit excellent thermal stability. At reaction temperatures around 70° C., the glycosyltransferase variants of the present disclosure all have specific activities of greater than 50 U/mg, representing a very significant improvement in specific activity and making them suitable for industrial-scale production.

The foregoing provides a detailed description of a glycosyltransferase for catalyzing production of Rebaudioside M (Reb M) and a recombinant gene thereof according to the present disclosure. Specific examples have been used herein to illustrate the principles and implementations of the present disclosure, and the descriptions of the above embodiments are merely intended to facilitate understanding of the methods and core concepts of the present disclosure. It should be noted that, for those skilled in the art, various modifications and alterations may be made to the present disclosure without departing from the principles of the present disclosure, and such modifications and alterations fall within the scope of the claims of the present disclosure.

1. A glycosyltransferase variant for catalyzing production of Rebaudioside M (Reb M), wherein the glycosyltransferase variant, as compared with a wild-type glycosyltransferase shown in SEQ ID NO: 1, comprises a combination of mutation sites K55N+N70T+S181P+S275R+K441E+T460K.

2. The glycosyltransferase variant of claim 1, wherein the glycosyltransferase variant has a mutation combination:
K55N+N70T+S181P+S275R+K441E+T460K+R414H+R90L+A112S+N50K+S193R+E3V+H156Y+T264S+R405P+D327E and has an amino acid sequence as set forth in SEQ ID NO: 9.

3. The glycosyltransferase variant of claim 1, wherein the glycosyltransferase variant has a mutation combination:
K55N+N70T+S181P+S275R+K441E+T460K+M146R+D327E+K224E and has an amino acid sequence as set forth in SEQ ID NO: 3.

4. The glycosyltransferase variant of claim 1, wherein the glycosyltransferase variant has a mutation combination:
K55N+N70T+S181P+S275R+K441E+T460K+K224E+194F+R414H+A112S and has an amino acid sequence as set forth in SEQ ID NO: 4.

5. The glycosyltransferase variant of claim 1, wherein the glycosyltransferase variant has a mutation combination:
K55N+N70T+S181P+S275R+K441E+T460K+194F+R90L+S242I+H156Y+A180V+R175H and has an amino acid sequence as set forth in SEQ ID NO: 5.

6. The glycosyltransferase variant of claim 1, wherein the glycosyltransferase variant has a mutation combination:
K55N+N70T+S181P+S275R+K441E+T460K+E3V+R414H+K224E+A112S+R90L+M146R+R298Q+A180V and has an amino acid sequence as set forth in SEQ ID NO: 6.

7. The glycosyltransferase variant of claim 1, wherein the glycosyltransferase variant has a mutation combination:
K55N+N70T+S181P+S275R+K441E+T460K+S193R+D100A+R298Q+K224E+R141P+R41 4H and has an amino acid sequence as set forth in SEQ ID NO: 7.

8. The glycosyltransferase variant of claim 1, wherein the glycosyltransferase variant has a mutation combination:
K55N+N70T+S181P+S275R+K441E+T460K+D100A+R414H+K224E+R405P+T264S+194F+R90L and has an amino acid sequence as set forth in SEQ ID NO: 8.

9. The glycosyltransferase variant of claim 1, wherein the glycosyltransferase variant has a mutation combination:
K55N+N70T+S181P+S275R+K441E+T460K+S193R+M146R+E3V and has an amino acid sequence as set forth in SEQ ID NO: 2.

10. The glycosyltransferase variant of claim 1, wherein the glycosyltransferase variant has a mutation combination:
K55N+N70T+S181P+S275R+K441E+T460K and has an amino acid sequence as set forth in SEQ ID NO: 10.

11. A method for preparing Rebaudioside M (Reb M), comprising:
mixing a substrate Rebaudioside D (Reb D) with the glycosyltransferase variants of claim 1 to react at a temperature from 50° C. to 75° C..

12. The method of claim 11, wherein the reaction is carried out at a temperature from 65° C. to 75° C..

13. A recombinant genetic material of a glycosyltransferase, wherein the recombinant genetic material is DNA or RNA capable of expressing any one of the glycosyltransferase variants of claim 1.

14. A recombinant strain of a glycosyltransferase, wherein the recombinant strain comprises the recombinant genetic material of claim 13.


SEQUENCE LISTING (10 sequences)

SEQ ID NO: 1 - protein, 461 aa, synthetic construct
MMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLTS

SEQ ID NO: 2 - protein, 461 aa, synthetic construct
MMVNKTETTVRRRRRIILFPVPFQGHINPILQLANVLYSKGFSITIFHTNFNKPNTSNYPHFTFRFILDTDPQDERISNLPTHGPLAGMRIPIINEHGADELRRELELLMLASEEDEEVSCLITDALWYFAQSVADSLNLRRLVLRTSSLFNFHAHVSLPQFDELGYLDPDDKTRLEEQAPGFPMLKVKDIKRAYSNWQILKEILGKMIKQTKASSGVIWNSFKELEESELETVIREIPAPSFLIPLPKHLTASSSSLLDHDRTVFQWLDQQPPRSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMEGGSSYESLESLVSYISSLKS

SEQ ID NO: 3 - protein, 461 aa, synthetic construct
MMENKTETTVRRRRRIILFPVPFQGHINPILQLANVLYSKGFSITIFHTNFNKPNTSNYPHFTFRFILDTDPQDERISNLPTHGPLAGMRIPIINEHGADELRRELELLMLASEEDEEVSCLITDALWYFAQSVADSLNLRRLVLRTSSLFNFHAHVSLPQFDELGYLDPDDKTRLEEQAPGFPMLKVKDIKSAYSNWQILKEILGKMIKQTKASSGVIWNSFEELEESELETVIREIPAPSFLIPLPKHLTASSSSLLDHDRTVFQWLDQQPPRSVLYVSFGSTSEVDEKDFLEIARGLVDSKQSFLWVVRPGFVKGSTWVEPLPEGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMEGGSSYESLESLVSYISSLKS

SEQ ID NO: 4 - protein, 461 aa, synthetic construct
MMENKTETTVRRRRRIILFPVPFQGHINPILQLANVLYSKGFSITIFHTNFNKPNTSNYPHFTFRFILDTDPQDERISNLPTHGPLAGMRIPIFNEHGADELRRELELLMLSSEEDEEVSCLITDALWYFAQSVADSLNLRRLVLMTSSLFNFHAHVSLPQFDELGYLDPDDKTRLEEQAPGFPMLKVKDIKSAYSNWQILKEILGKMIKQTKASSGVIWNSFEELEESELETVIREIPAPSFLIPLPKHLTASSSSLLDHDRTVFQWLDQQPPRSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRHVMVDEEGEYIRQNARVLKQKADVSLMEGGSSYESLESLVSYISSLKS

SEQ ID NO: 5 - protein, 461 aa, synthetic construct
MMENKTETTVRRRRRIILFPVPFQGHINPILQLANVLYSKGFSITIFHTNFNKPNTSNYPHFTFRFILDTDPQDERISNLPTHGPLAGMLIPIFNEHGADELRRELELLMLASEEDEEVSCLITDALWYFAQSVADSLNLRRLVLMTSSLFNFHAYVSLPQFDELGYLDPDDKTHLEEQVPGFPMLKVKDIKSAYSNWQILKEILGKMIKQTKASSGVIWNSFKELEESELETVIREIPAPIFLIPLPKHLTASSSSLLDHDRTVFQWLDQQPPRSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMEGGSSYESLESLVSYISSLKS

SEQ ID NO: 6 - protein, 461 aa, synthetic construct
MMVNKTETTVRRRRRIILFPVPFQGHINPILQLANVLYSKGFSITIFHTNFNKPNTSNYPHFTFRFILDTDPQDERISNLPTHGPLAGMLIPIINEHGADELRRELELLMLSSEEDEEVSCLITDALWYFAQSVADSLNLRRLVLRTSSLFNFHAHVSLPQFDELGYLDPDDKTRLEEQVPGFPMLKVKDIKSAYSNWQILKEILGKMIKQTKASSGVIWNSFEELEESELETVIREIPAPSFLIPLPKHLTASSSSLLDHDRTVFQWLDQQPPRSVLYVSFGSTSEVDEKDFLEIAQGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRHVMVDEEGEYIRQNARVLKQKADVSLMEGGSSYESLESLVSYISSLKS

SEQ ID NO: 7 - protein, 461 aa, synthetic construct
MMENKTETTVRRRRRIILFPVPFQGHINPILQLANVLYSKGFSITIFHTNFNKPNTSNYPHFTFRFILDTDPQDERISNLPTHGPLAGMRIPIINEHGAAELRRELELLMLASEEDEEVSCLITDALWYFAQSVADSLNLPRLVLMTSSLFNFHAHVSLPQFDELGYLDPDDKTRLEEQAPGFPMLKVKDIKRAYSNWQILKEILGKMIKQTKASSGVIWNSFEELEESELETVIREIPAPSFLIPLPKHLTASSSSLLDHDRTVFQWLDQQPPRSVLYVSFGSTSEVDEKDFLEIAQGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRHVMVDEEGEYIRQNARVLKQKADVSLMEGGSSYESLESLVSYISSLKS

SEQ ID NO: 8 - protein, 461 aa, synthetic construct
MMENKTETTVRRRRRIILFPVPFQGHINPILQLANVLYSKGFSITIFHTNFNKPNTSNYPHFTFRFILDTDPQDERISNLPTHGPLAGMLIPIFNEHGAAELRRELELLMLASEEDEEVSCLITDALWYFAQSVADSLNLRRLVLMTSSLFNFHAHVSLPQFDELGYLDPDDKTRLEEQAPGFPMLKVKDIKSAYSNWQILKEILGKMIKQTKASSGVIWNSFEELEESELETVIREIPAPSFLIPLPKHLTASSSSLLDHDRSVFQWLDQQPPRSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWEPGEIANAIRHVMVDEEGEYIRQNARVLKQKADVSLMEGGSSYESLESLVSYISSLKS

SEQ ID NO: 9 - protein, 461 aa, synthetic construct
MMVNKTETTVRRRRRIILFPVPFQGHINPILQLANVLYSKGFSITIFHTKFNKPNTSNYPHFTFRFILDTDPQDERISNLPTHGPLAGMLIPIINEHGADELRRELELLMLSSEEDEEVSCLITDALWYFAQSVADSLNLRRLVLMTSSLFNFHAYVSLPQFDELGYLDPDDKTRLEEQAPGFPMLKVKDIKRAYSNWQILKEILGKMIKQTKASSGVIWNSFKELEESELETVIREIPAPSFLIPLPKHLTASSSSLLDHDRSVFQWLDQQPPRSVLYVSFGSTSEVDEKDFLEIARGLVDSKQSFLWVVRPGFVKGSTWVEPLPEGFLGERGRIVKWVPQQEVLAHGAIGAFWTHSGWNSTLESVCEGVPMIFSDFGLDQPLNARYMSDVLKVGVYLENGWEPGEIANAIRHVMVDEEGEYIRQNARVLKQKADVSLMEGGSSYESLESLVSYISSLKS

SEQ ID NO: 10 - protein, 461 aa, synthetic construct
MMENKTETTVRRRRRIILFPVPFQGHINPILQLANVLYSKGFSITIFHTNFNKPNTSNYPHFTFRFILDTDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQAPGFPMLKVKDIKSAYSNWQILKEILGKMIKQTKASSGVIWNSFKELEESELETVIREIPAPSFLIPLPKHLTASSSSLLDHDRTVFQWLDQQPPRSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMEGGSSYESLESLVSYISSLKS